(19) United States
(12) Patent Application Publication
(10) Pub. No.: US 2023/0302446 A1
(43) Pub. Date: Sep. 28, 2023

(54) CARTRIDGE FOR DETECTING TARGET ANALYTE

(71) Applicant: SEEGENE, INC., Seoul (KR)

(72) Inventors: Phillip HALBERT, San Francisco, CA (US); Aaron Matarese, Cranston, RI (US)

(73) Assignee: SEEGEME. INC., Seoul (KR)

(21) Appl. No.: 17/704,557

(22) Filed: Mar. 25, 2022

(51) Int. Cl. B01L 3/00; C12Q 1/6806

(52) U.S. Cl. CPC B01L 3/502715; C12Q 1/6806; B01L 2400/0694; B01L 2300/0681

(57) ABSTRACT

A cartridge for detecting a target analyte according to an embodiment of the present disclosure comprises a sample containing part in which a sample is receivable; a sample drawing part in which the sample is drawn up into a pipette tip; and a sample flowing channel via which the sample containing part and the sample drawing part are in fluid communication with each other, wherein a sample flows into a pipette tip from the sample containing part via the sample flowing channel by a negative pressure delivered through the pipette tip entering the sample drawing part in a state where the pipette tip is in close contact with an outlet of the sample flowing channel.

### TECHNICAL FIELD

The present disclosure relates to a cartridge for detecting a target analyte.

### BACKGROUND ART

Nowadays people's interest in health increases and life expectancy extends. Thus, accurate analysis of pathogens and in vitro nucleic acid-based molecular diagnosis such as genetic analysis for a patient become significant, and the demand therefor is on the rise. Nucleic acid-based molecular diagnosis is performed by extracting nucleic acids from a sample and confirming whether a target nucleic acid is present in the extracted nucleic acids.

Polymerase chain reaction (PCR) is the most widely used nucleic acid amplification method, and the PCR process is performed by repeated cycling including denaturation of double-stranded DNA, annealing of oligonucleotide primers to the DNA templates and extension of primers by DNA polymerase (Mullis et al.; U.S. Pat. Nos. 4,683,195, 4,683,202 and 4,800,159; Saiki et al., (1985) Science 230, 1350-1354).

Real-time PCR using a fluorescent material is a method of detecting an increase in fluorescence intensity according to nucleic acid amplification during the PCR process. Real-time PCR enables multiplex detection by using different fluorescent dyes for each target; however, the technique requires expensive equipment and a lot of time for detection.

Meanwhile, recently, point of care testing which diagnoses patient's diseases quickly and correctly at any time and any place draws attention as a very significant technique of evidence-based precision health.

However, considering the characteristics of POC testing equipment which requires quick processing time, while its structure is to be streamlined and made in a compact size, a sample should be subjected to separate filtration before being injected in a cartridge. Such additional filtration requires time and labor forces.

### DISCLOSURE

### Technical Problem

The present disclosure provides a cartridge for detecting a target analyte which uses a crude sample that is not filtered by injecting the sample itself in a cartridge.

### Technical Solution

An embodiment of the present disclosure provides a cartridge for detecting a target analyte, comprising a sample containing part in which a sample is receivable; a sample drawing part in which the sample is drawn up into a pipette tip; and a sample flowing channel via which the sample containing part and the sample drawing part are in fluid communication with each other, wherein a sample flows into a pipette tip from the sample containing part via the sample flowing channel by a negative pressure delivered through the pipette tip entering the sample drawing part in a state where the pipette tip is in close contact with an outlet of the sample flowing channel.

The sample may flow directly into the pipette tip from the sample flowing channel.

The sample drawing part may have a pipette tip support part for supporting the end of the pipette tip.

The pipette tip support part may have a pipette tip support hole where the end of the pipette tip is inserted into or in close contact with.

The pipette tip support part may have a sloping surface sloping downwards towards the pipette tip support hole.

The sloping surface of the pipette tip support part may have a conical shape.

The pipette tip support hole may be the outlet of the sample flowing channel.

An inlet of the pipette tip and an inside of the sample drawing part may be fluidly disconnected from each other in a state where the pipette tip is inserted into the pipette tip support hole or is in close contact with the pipette tip support hole.

The negative pressure delivered through the pipette tip may be delivered directly to the sample flowing channel in a state where the pipette tip is inserted into the pipette tip support hole or is in close contact with the pipette tip support hole.

The sample flowing channel may fluidly connect a lower part of the sample containing part with a lower part of the sample drawing part.

The sample drawing part may have a pipette tip support hole where the end of the pipette tip is inserted into or in close contact with, and the sample flowing channel may be fluidly coupled to the pipette tip support hole.

The sample containing part or the sample flowing channel may further comprise a filter member.

The filter member may comprise a part spaced away from a bottom surface of the sample containing part by a certain distance.

The sample containing part may have a pipette tip entrance that is prepared as a one-way valve.

When a pipette leaves the pipette tip entrance formed in the sample containing part, the one-way valve may be closed to fluidly disconnect an inside of the sample containing part from an outside thereof.

The cartridge may further comprise a separation wall for dividing a first chamber of the sample containing part and a second chamber of the sample drawing part, and the sample flowing channel may pass through the separation wall or pass below the separation wall.

The cartridge may further comprise a filter member disposed in front of an inlet of the sample flowing channel of the first chamber.

The filter member may comprise a part spaced away from a bottom surface of the first chamber by a certain distance, and the sample flowing channel may be in fluid communication with a space below the filter member.

The first chamber may further comprise a spacer for supporting the filter member.

In the filter member, an area that is supported on the spacer may be smaller than an area that is not supported on the spacer.

The spacer may extend from a surface of the first chamber except from a separation wall in which an inlet of the sample flowing channel is formed.

A width of the sample flowing channel may be smaller than a width of the separation wall, and an inlet of the sample flowing channel may become narrower in a flow direction of a fluid.

The cartridge may further comprise a housing comprising the first chamber, the second chamber and the separation wall, wherein a lower surface of the first chamber and the sample flowing channel is opened, and a housing sealing member for sealing the opened lower surface of the housing.

The housing sealing member may be an elastically deformable membrane.

An embodiment of the present disclosure provides a cartridge for detecting a target analyte, comprising a body part including a pretreatment area having a plurality of wells, for pretreating a sample, and a detection area for detecting whether a target analyte is present in the sample pretreated in the pretreatment area, wherein the body part comprises a sample containing well in which a crude sample is receivable, the sample containing well having a filter member; a sample drawing well in which the sample is drawn up into a pipette tip; a plurality of buffer wells in which buffers are receivable; at least one reagent well in which reagent used for extraction or detection is receivable; a processing well in which mixing of different fluids or extraction occurs; and at least one detection well which is in communication with the pretreatment area via a channel, wherein the sample containing well and the sample drawing well are in fluid communication with each other via a sample flowing channel and wherein the sample flows directly into a pipette tip from the sample containing well via the sample flowing channel by a negative pressure delivered through the pipette tip entering the sample drawing well.

The cartridge may further comprise a sample inlet well in which the crude sample comes in, and a waste well in which debris is stored.

The pipette tip entering the sample drawing well may suck a sample filtered by passing through the filter member from the sample containing well in which the sample is receivable.

### Advantageous Effects

A cartridge according to an embodiment of the present disclosure uses a crude sample that was not subjected to filtration and has a filtering system therein, thus obtaining highly accurate result of detection of a target analyte.

Also, a cartridge according to an embodiment of the present disclosure separates a sample containing part from a sample drawing part and has a filter, thus obtaining a sample that is filtered.

Also, a cartridge according to an embodiment of the present disclosure applies a negative pressure delivered through a pipette tip directly to the sample containing part, allowing a sample to pass through a filter quickly.

Also, a cartridge according to an embodiment of the present disclosure employs a structure in which part of the end of the pipette tip is inserted into a pipette tip support hole in the sample drawing part to suck a sample, thus drawing up a sample while minimizing the residue that is left in the sample drawing part.

Also, a cartridge according to an embodiment of the present disclosure prevents an end of the pipette tip from touching the bottom of the sample drawing part, thus preventing damage and contamination of the end of the pipette tip.

The effects of the present disclosure are not limited to the above-mentioned effects, and it should be understood that the effects of the present disclosure include all effects that could be inferred from the configuration of the disclosure described in the detailed description of the disclosure or the appended claims.

### DESCRIPTION OF DRAWINGS

FIG. 1 is a perspective view illustrating a cartridge according to an embodiment of the present disclosure;

FIG. 2 is a top plane view illustrating a cartridge according to an embodiment of the present disclosure;

FIG. 3 is a cross-sectional view taken along line A-A in FIG. 2;

FIG. 4 is an exploded perspective view of FIG. 3;

FIG. 5 shows a difference in sample suction depending on whether there is a pipette tip support hole; and

FIG. 6 is a cross-sectional view illustrating a cartridge according to another embodiment of the present disclosure.

### MODE FOR INVENTION

Hereinafter, the present disclosure will be explained with reference to embodiments and example drawings. The embodiments are for illustrative purposes only, and it should be apparent to a person having ordinary knowledge in the art that the scope of the present disclosure is not limited to the embodiments.

In addition, in adding reference numerals to the components of each drawing, it should be noted that same reference numerals are assigned to same components as much as possible even though they are shown in different drawings. In addition, in describing the embodiments of the present disclosure, when it is determined that a detailed description of a related well-known configuration or function interferences with the understanding of the embodiments of the present disclosure, the detailed description thereof will be omitted.

In addition, in describing the components of the embodiments of the present disclosure, terms such as first, second, A, B, (a), (b), (i), (ii), etc. may be used. These terms are only for distinguishing the components from other components, and the nature or order of the components is not limited by the terms. When a component is described as being “connected,” “coupled” or “fastened” to other component, the component may be directly connected or fastened to the other component, but it will be understood that another component may be “connected,” “coupled” or “fastened” between the components.

The present disclosure relates to an apparatus for detecting a target analyte in a sample.

As used herein, the term “sample” may include a biological sample (e.g., cells, tissues and fluids from a biological source) and a non-biological sample (e.g., food, water and soil). Examples of the biological sample may include viruses, bacteria, tissues, cells, blood (e.g., whole blood, plasma and serum), lymph, bone marrow fluid, salvia, sputum, swab, aspiration, milk, urine, feces, ocular fluid, semen, brain extract, spinal fluid, joint fluid, thymus fluid, bronchoalveolar lavage fluid, ascites and amniotic fluid. Also, the sample may include natural nucleic acid molecules isolated from a biological source and synthetic nucleic acid molecules. According to an embodiment of the present disclosure, the sample may include an additional substance such as water, deionized water, saline solution, pH buffer, acid solution or alkaline solution.

A target analyte refers to a substance that is the subject of analysis. The analysis may mean obtaining information on, for example, the presence, amount, concentration, sequence, activity or property of the analyte in the sample. The analyte may include various substances (e.g., biological substance and non-biological substance such as compounds). Specifically, the analyte may include a biological substance such as nucleic acid molecules (e.g., DNA and RNA), proteins, peptides, carbohydrates, lipids, amino acids, biological compounds, hormones, antibodies, antigens, metabolites or cells. According to an embodiment of the present disclosure, the analyte may be nucleic acid molecules.

The apparatus for detecting a target analyte of the present disclosure may be an apparatus for detecting a target nucleic acid. The apparatus for detecting a target nucleic acid allows a nucleic acid reaction to be performed in a sample, to detect a target nucleic acid.

The nucleic acid reaction refers to sequential physical and chemical reactions which generate a signal depending on the presence of a nucleic acid of a specific sequence in the sample or the amount thereof. The nucleic acid reaction may include the binding of a nucleic acid of a specific sequence in a sample to other nucleic acids or substances, or replication, cleavage or decomposition of a nucleic acid of a specific sequence in the sample. The nucleic acid reaction may involve a nucleic acid amplification reaction. The nucleic acid amplification reaction may include amplification of a target nucleic acid. The nucleic acid amplification reaction may specifically amplify the target nucleic acid.

The nucleic acid reaction may a signal-generation reaction which can generate a signal depending on the presence/absence of a target nucleic acid in the sample or the amount thereof. The signal-generation reaction may be a technique of genetic analysis such as PCR, real-time PCR or microarray.

Various methods for generating an optical signal which indicates the presence of a target nucleic acid using a nucleic acid reaction are known. Representative examples thereof include the following: TaqMan™ probe method (U.S. Pat. No. 5,210,015), molecular beacons method (Tyagi et al., Nature Biotechnology v.14 Mar. 1996), scorpion method (Whitcombe et al., Nature Biotechnology 17:804-807(1999)), sunrise or amplifluor method (Nazarenko et al., 2516-2521 Nucleic Acids Research, 25(12):2516-2521(1997), and U.S. Pat. No. 6,117,635), lux method (U.S. Pat. No. 7,537,886), CPT (Duck P, et al., Biotechniques, 9:142-148(1990)), LNA method (U.S. Pat. No. 6,977,295), plexor method (Sherrill C B, et al, Journal of the American Chemical Society, 126:4550-4556(2004)), Hybeacons™ (D. J. French, et al., Molecular and Cellular Probes (2001) 13, 363-374 and U.S. Pat. No. 7,348,141), dual-labeled, self-quenched probe (U.S. Pat. No. 5,876,930), hybridization probe (Bernard P S, et al., Clin Chem 2000, 46, 147-148), PTOCE (PTO cleavage and extension) method (WO 2012/096523), PCE-SH (PTO Cleavage and Extension-Dependent Signaling Oligonucleotide Hybridization) method (WO 2013/115442), PCE-NH (PTO Cleavage and Extension-Dependent Non-Hybridization) method (PCT/KR2013/012312) and CER method (WO 2011/037306).

An apparatus for detecting a target analyte according to an embodiment of the present disclosure may be an apparatus for detecting a nucleic acid, and may detect a signal generated depending on the presence of the target nucleic acid. The apparatus for detecting a nucleic acid may amplify and detect a signal with nucleic acid amplification. Alternatively, the apparatus for detecting a nucleic acid may amplify and detect a signal without nucleic acid amplification. Preferably, the apparatus for detecting a nucleic acid detects a signal with nucleic acid amplification.

An apparatus for detecting a target analyte according to an embodiment of the present disclosure may comprise a nucleic acid amplifier.

A nucleic acid amplifier refers to an apparatus for performing a nucleic acid amplification reaction which amplifies a nucleic acid having a specific nucleotide sequence. Examples of the method for amplifying a nucleic acid include polymerase chain reaction (PCR), ligase chain reaction (LCR) (U.S. Pat. Nos. 4,683,195 and 4,683,202; PCR Protocols: A Guide to Methods and Applications (Innis et al., eds, 1990)), strand displacement amplification (SDA) (Walker, et al. Nucleic Acids Res. 20(7):1691-6 (1992); Walker P C R Methods Appl 3(1):1-6 (1993)), transcription-mediated amplification (Phyffer, et al., J. Clin. Microbiol. 34:834-841 (1996); Vuorinen, et al., J. Clin. Microbiol. 33:1856-1859 (1995)), nucleic acid sequence-based amplification (NASBA) (Compton, Nature 350(6313):91-2 (1991)), rolling circle amplification (RCA) (Lisby, Mol. Biotechnol. 12(1):75-99 (1999); Hatch et al., Genet. Anal. 15(2):35-40 (1999)) and Q-beta Replicase (Lizardi et al., BiolTechnology 6:1197 (1988)), etc.

An apparatus for detecting a target analyte according to an embodiment of the present disclosure may be an apparatus for performing a nucleic acid amplification reaction with temperature changes. For example, the nucleic acid amplifier may carry out a denaturing step, an annealing step and an extension (or elongation) step to amplify deoxyribonucleic acid (DNA) having a specific base sequence.

In the denaturing step, a sample and reagent solution containing double-stranded DNA templates is heated to a specific temperature, for example about 95° C., to separate double-stranded DNA into single-stranded DNA. In the annealing step, an oligonucleotide primer having a nucleotide sequence complementary to the nucleotide sequence of a nucleic acid to be amplified is provided, and the primer and the separated single-stranded DNA are cooled down to a specific temperature, for example 60° C., to promote the primer binding to the specific nucleotide sequence of the single-stranded DNA to form a partial DNA-primer complex. In the extension step, the solution is maintained at a specific temperature, for example 72° C., after the annealing step to form double-stranded DNA by DNA polymerase based on the primer of the partial DNA-primer complex.

The aforementioned three steps are repeated, for example 10 to 50 times, geometrically amplifying DNA having the specific nucleotide sequence. In some cases, the nucleic acid amplifier may perform the annealing step and extension step simultaneously. In this case, the nucleic acid amplifier may complete one cycle by performing two steps including a denaturing step and an annealing/extension step.

Particularly, an apparatus for detecting a target analyte according to an embodiment may be an apparatus for performing a nucleic acid amplification reaction with temperature changes and a reaction of generating an optical signal depending on the presence of a nucleic acid and detecting the generated optical signal.

Also, an apparatus for detecting a target analyte according to an embodiment may comprise an optical module, a thermal module, a controller for controlling the modules, a frame for supporting the optical module and thermal module, a case for enclosing the components and a display unit.

The optical module may comprise an optical housing for forming a dark room, a light source for generating excitation light, an excitation light filter unit for providing a wavelength range of a target analyte, a beam splitter for setting a light path and a detection unit for detecting emission light from a sample. The optical module may further comprise a heat lead for pressing and heating a cover of a reaction vessel (e.g., tube or cartridge) accommodated in a thermal block.

The excitation light filter unit may comprise an excitation light filter wheel having a plurality of filters with different wavelength ranges and a drive unit for driving the excitement light filter wheel.

The detection unit may comprise a detection sensor for obtaining images from emission light, an emission light filter unit and an emission light lens array for adjusting the light path and focus.

The emission light filter unit may comprise an emission light filter wheel having a plurality of filters with different wavelength ranges and a drive unit for driving the emission light filter wheel.

The thermal module may comprise a thermal block in which a reaction vessel is accommodated or a reaction vessel is seated, a thermal unit comprising a thermal element, a cooling unit for cooling the thermal block and a thermal block housing.

The cooling unit may comprise a heat radiating plate and a cooling fan.

Next, the operation of an apparatus for detecting a target analyte according to an embodiment is described.

A light source emits light to excite a fluorescent substance included in a sample. An example of the light source may be a light emitting diode (LED).

The light emitted from the light source may be indicated as an excitation beam, and the light emitted from the sample may be indicated as an emission beam. The path of the excitation beam emitted from the light source may be indicated as an excitation path, and the path of the emission beam emitted from the sample may be indicated as an emission path.

A beam splitter may selectively reflect or transmit incident light. The excitation light of the light source penetrates the beam splitter and passes through a hole of a heat lead to arrive at a reaction vessel accommodated in a thermal block.

The light emitted from the sample is reflected at the beam splitter and passes through an emission light lens array to arrive at a detection sensor.

A thermal block may be a thermally conductive material. When the thermal block is in contact with reaction vessels, heat may be transferred from the thermal block to the reaction vessels. The thermal block may be made of, for example, metal such as aluminum, gold, silver, nickel or copper.

A thermal block housing may accommodate the thermal block, thermal element, cooling unit, etc.

A thermal element may increase or decrease the temperature of the thermal block. The thermal element is disposed below the thermal block and is in contact with the thermal block to transfer heat to the thermal block or absorb heat from the thermal block. For example, the thermal element may be a Peltier element or a heating wire, and comprise an FPCB to which the element is connected.

A cooling unit may be disposed below the thermal block, and comprise a heat radiating plate or a heat radiating fin for emitting heat to the outside of the thermal block. Also, the cooling unit may comprise a cooling fan for providing the air to cool the heat radiating plate or heat radiating fin.

A detection unit detects a signal from the sample. Specifically, the detection unit comprises a detection sensor for detecting fluorescence generated from the sample. The detection sensor may be a charge coupled device (CCD), complementary metal oxide semiconductor (CMOS), field effect transistor or photodiode.

An apparatus for detecting a target analyte according to an embodiment comprises a sample holder, a light emitting module and a detection module.

A sample refers to a substance in which a target analyte is to be detected. The sample includes a biological sample (e.g., cells, tissues and fluids from a biological source) and a non-biological sample (e.g., food, water and soil). Examples of the biological sample include, but are not limited to, viruses, bacteria, tissues, cells, blood, plasma, serum, lymph, sputum, swab, aspiration, bronchoalveolar lavage fluid, milk, urine, feces, ocular fluid, salvia, semen, brain extract, spinal fluid (SCF), extracts of appendix, spleen and tonsil tissues, ascites and amniotic fluid. Also, the sample may include natural nucleic acid molecules isolated from a biological source and synthetic nucleic acid molecules.

The sample according to the present disclosure may include a substance necessary for detecting a target analyte. For example, the sample may include an additional substance such as water, deionized water, saline solution, pH buffer, acid solution or alkaline solution. According to an embodiment of the present disclosure, the sample may include an optical probe. The optical probe refers to a probe which generates an optical signal depending on the presence of a target nucleic acid. The optical probe may be a fluorescent probe. The fluorescent probe as used herein may include any molecules known in the art.

The light emitting module according to the present disclosure supplies a proper optical stimulus to a sample contained in the sample holder, and the detection module senses an optical signal generated from the sample in response thereto.

The optical signal may be a luminescence signal, phosphorescence signal, chemiluminescence signal, fluorescence signal, polarized fluorescence signal or other colored signal. The optical signal may be generated in response to an optical stimulus given to the sample.

The sample holder has a sample containing part for containing the sample. The sample holder is a component for containing the sample directly in the sample containing part or accommodating a reaction vessel containing the sample.

As used herein, the expression “the sample holder may contain the sample” may be used to comprehensively include the cases where the sample holder contains the sample directly in the sample containing part or accommodates the reaction vessel containing the sample.

The sample holder allows the sample to be placed in a predetermined position, such that an optical stimulus from a light emitting module arrives at the sample and an optical signal generated from the sample arrives at a detection module.

Heat may be supplied to the sample holder by a heat generating element, and heat is transferred to the sample contained directly in the sample holder or the sample contained in the reaction vessel.

The reaction vessel may be made of various materials, for example, plastic, ceramic, glass or metal.

The sample holder for accommodating a reaction vessel may be in the shape of a block or plate. The sample holder for accommodating a reaction vessel may include a recess, for example a well, for accommodating the reaction vessel or have a flat surface. The sample holder for accommodating a reaction vessel may have a structure of guiding the position of the reaction vessel or fixing the reaction vessel thereto.

A sample holder is configured to contain at least one sample.

One of the representative examples of the sample holder for accommodating a reaction vessel is a heat block. The heat block includes a plurality of wells or holes, and reaction vessels may be accommodated in the wells or holes.

The sample holder accommodating sample reaction vessels means a state in which sample reaction vessels are placed in a plurality of wells of the sample holder or are arranged in assigned positions on the sample holder.

A reaction vessel is used for containing a sample to be analyzed, and includes vessels of a variety of shapes, for example a tube, vial, strip in which a plurality of single tubes are connected, plate in which a plurality of tubes are connected, microcard, chip, cuvette or cartridge.

The sample holder for directly containing the sample may have a shape of the reaction vessel as described above, and be made of the material of the reaction vessel as described above.

According to an embodiment, the sample holder may be made of a material having a thermal conductivity. When the sample holder is in direct contact with the sample or is in contact with reaction vessels, heat may be transferred to the sample or the sample in the reaction vessels from the sample holder.

The sample holder may be made of metal such as aluminum, gold, silver, nickel or copper, plastic or ceramic.

The sample holder is configured to contain a plurality of samples, and adjusts the temperature of the plurality of samples to cause a reaction for detection such as a nucleic acid amplification reaction. For example, in the case where the sample holder is a heat block having a plurality of wells, the sample holder is formed as one heat block, and each well of the heat block may be configured not to be thermally independent from each other. In this case, all wells of the sample holder in which the samples are contained have the same temperature, and the temperatures of the contained samples cannot be adjusted independently according to different protocols.

Alternatively, the sample holder may be configured to adjust the temperatures of some of the samples in the sample holder according to different protocols. The sample holder may have two or more reaction regions that are thermally independent from each other. Each of the reaction regions is thermally independent. Heat is not transferred from one reaction region to other reaction regions. An insulating material or an air gap may be present between the reaction regions. The temperatures of the reaction regions may be controlled independently. Reaction protocols including temperature and time may be set individually for each of the reaction regions, and each of the reaction regions may perform a reaction according to the individually set protocols. As such, reactions are performed in the reaction regions according to the individually set protocols, and thus the time points of detecting light in the reaction regions are independent from each other.

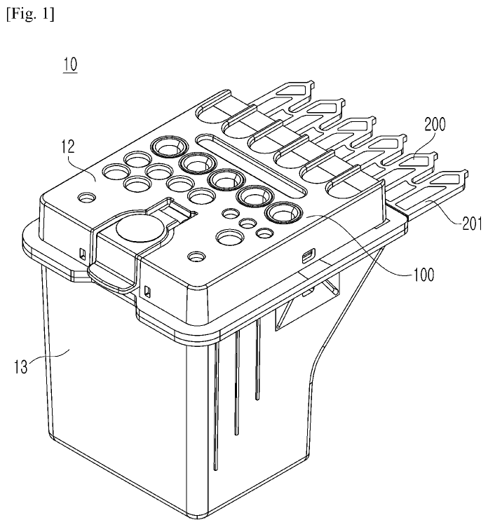

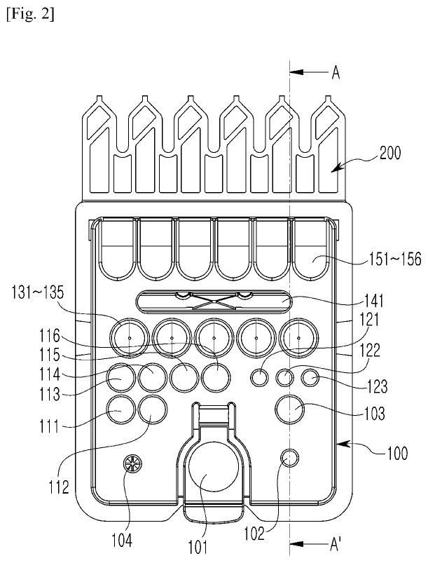

FIG. 1 is a perspective view illustrating a cartridge according to an embodiment of the present disclosure, and FIG. 2 is a top plane view illustrating a cartridge according to an embodiment of the present disclosure.

A cartridge 10 for detecting a target analyte according to an embodiment may comprise a body part at one side of which a sample, beads or reagents are contained, for performing detection of a target analyte, an upper case 12 coupled to an upper part of the body part and a lower case 13 coupled to a lower part of the body part, for accommodating wells inside. The body part may not be shown in the drawings by being covered with the upper case 12 and the lower case 13.

The body part may have a pretreatment area 100 having a plurality of wells, for pretreating samples, and a detection area 200 which is in fluid communication with detection wells 151-156, which is the final stage of the pretreatment area 100, to detect a target analyte. The detection area 200 extends in the lateral direction from one side of the pretreatment area 100.

The pretreatment area 100 may be in the shape of a block, having openings of the plurality of wells on an upper surface. The wells may extend downwards. Each well may have different shapes and depths.

The pretreatment area 100 may further comprise pipette tip holding holes 131-135 for holding a plurality of pipette tips. The pipette tip holding holes 131-135 may be prepared as an opening on the upper surface of the body part. The pipette tip holding hole 131-135 may have an opening diameter smaller than an opening diameter of the top of the pipette tip having the shape of a cone, allowing the top edge of the pipette tip to be seated on the pipette tip holding hole 131-135.

The detection area 200 may have a detection channel 201 which is in fluid communication with the detection wells 151-156 of the pretreatment area 100. The detection channel 201 may protrude outwards from the upper case 12 and lower case 13 to be placed on a light path of a detection module (not shown).

The upper case 12 may comprise an opening for covering the top of the pretreatment area 100, in correspondence to the openings of the wells. For example, some of the openings of the body part may be sealed with a film or membrane, and the film or membrane may be exposed through the opening of the upper case 12.

The lower case 13 may be configured to accommodate the wells and pipette tips that are held in the pipette tip holding holes 131-135. The lower case 13 may protect the wells and pipette tips from an external impact.

The wells may be arranged side by side in the X-axis direction in an XY plane of the pretreatment area 100 to provide a row, and a plurality of rows may be arranged in the Y-axis direction in the XY plane. The pipette tip holding holes 131-135 may be arranged side by side in the X-axis direction to provide a row.

The openings of the wells may be in a variety of shapes such as a circle, quadrangle or oval. Among the wells, wells performing the same function may have the same shape and depth, and wells performing different functions may have different shapes or depths.

The wells may include a first sample containing well 101 in which a sample tube containing a crude sample is seated, a second sample containing well 102 in which part of the sample transferred from the first sample containing well 101 is contained for sample filtering, a waste well 104 in which debris is stored, a sample drawing well 103 in which the sample which is filtered after being transferred to the second sample containing well 102 is drawn, buffer wells 112-116 in which buffers are contained, bead wells 121-123 in which beads are contained, a processing well 141 in which fluids are mixed or extracted, and detection wells 151-156 which are in communication with the detection channel.

Specifically, the waste well 104, first sample containing well 101 and second sample containing well 102 may be arranged in the first row. The sample drawing well 103 and some of the buffer wells 112-116 may be arranged in the second row. The rest of the buffer wells 112-116 and the bead wells 121-123 may be arranged in the third row. The pipette tip holding holes 131-135 may be arranged in the fourth row. The processing well 141 may be arranged in the fifth row. The detection wells 151-156 may be arranged in the sixth row.

The openings of the wells may have a sealing membrane that is penetrable by a force applied from a pipette tip or a one-way valve that allows a sample to flow inside the wells but prevents a sample from flowing outside the wells. The sealing membrane may be a sealing foil.

The first sample containing well 101 may contain a crude sample which includes impurities or is not pretreated. The second sample containing well 102 may contain the sample that is drawn from the first sample containing well 101 and injected by a pipette. The second sample containing well 102 may be sealed with a sealing membrane or covered with a one-way valve. The sealing membrane may be a sealing foil.

The one-way valve may comprise a plurality of pieces which are elastically deformable inwards of the well, and the plurality of pieces may be arranged in the circumferential direction of the second sample containing well 102.

In the case where the opening of the second sample containing well 102 is sealed with a sealing membrane, the sealing membrane is broken when the pipette enters. In the case where the opening of the second sample containing well 102 is covered with a one-way valve, the one-way valve is opened when the pipette enters and the one-way valve is closed when the pipette leaves upon completion of sample injection.

The second sample containing well 102 may have a filter structure to remove debris, etc. from the crude sample, and the sample from which debris is removed may flow from the second sample containing well 102 to the sample drawing well 103.

The cartridge 10 for detecting a target analyte according to an embodiment of the present disclosure may use a crude sample or a sample that has been filtered outside. When using a filtered sample, the sample may be put into the first sample containing well 101 and a determined amount of the sample may be drawn up from the first sample containing well 101 using a pipette. When using a crude sample, the sample may be put into the second sample containing well 102 and a determined amount of the sample may be drawn up from the sample drawing well 103. A negative pressure created by the pipette may help the sample flow between the second sample containing well 102 and the sample drawing well 103. This will be described in detail below.

For the buffer wells, an elution buffer well 112 in which an elution buffer is contained and a dummy well 111 as an extra may be arranged in the second row, and a lysis buffer well 113 in which a lysis buffer is contained and a binding buffer well 114 in which a binding buffer is contained, and a plurality of buffer wells 115, 116 in which cleaning buffers are contained may be arranged at one side of the third row.

For the bead wells, a first bead well 121 in which proteinase K beads are contained, a second bead well 122 in which internal control beads are contained and a third bead well 123 in which magnetic beads are contained may be arranged at the other side of the third row.

Or, at least one bead well may be a reagent well in which reagent used for extraction or detection is receivable.

Five pipette tip holding holes 131-135 may be arranged in the fourth row, and pipette tips may be held in the pipette tip holding holes, respectively.

The processing well 141 may be arranged in the fifth row. The processing well 141 may be in an elongated shape in the row direction. The processing well 141 may be sealed with a sealing membrane or covered with a one-way valve. The one-way valve may comprise a plurality of pieces which are elastically deformable inwards of the well, and the plurality of pieces may be arranged in the circumferential direction of the processing well 141.

In the case where the opening of the processing well 141 is sealed with a sealing membrane, the sealing membrane is broken when the pipette enters. In the case where the opening of the processing well 141 is covered with a one-way valve, the one-way valve is opened when the pipette enters and the one-way valve is closed when the pipette leaves upon completion of sample injection.

Six detection wells 151-156 may be arranged in the sixth row. A lyophilized mastermix may be contained in each detection well. Each of the detection wells 151-156 may be connected with a detection site. For example, when a solution is injected into the detection wells 151-156, the lyophilized mastermix may be dissolved, and the solution in which the mastermix is dissolved may move to the detection site along a channel. The detection site has a light transmissive area and detects a target analyte using a detector.

Meanwhile, a positive pressure may be applied to the detection wells 151-156 for the solution in which the mastermix is dissolved to move to the detection site. For example, a manifold device linked with a pump may be connected to the detection wells 151-156. When the pump operates, a positive pressure delivered to the detection wells 151-156 pushes the solution to the detection site.

Hereinafter, a process for detecting a target analyte using a cartridge according to an embodiment is described.

A robot of an apparatus for detecting a target analyte may await a user input before performing a protocol. For example, after placing a cartridge 10 in an inlet of the apparatus for detecting a target analyte, a user may send an operation signal for the robot to perform a protocol, or the robot may perform a protocol after a predetermined period of time without a user's operation signal.

A protocol performed by the robot is as follows.

The robot of the apparatus for detecting a target analyte moves a pipette to be coupled to a first pipette tip held in a first pipette tip holding hole 131.

The robot moves the pipette to which the pipette tip is coupled to suck the lysis buffer in the lysis buffer well 114 and transfer the buffer to the first bead well 121 in which proteinase K beads are contained. Then, the robot draws up the lysis buffer in the lysis buffer well 114 and transfers the buffer to the second bead well 122 in which internal control beads are contained.

Then, the proteinase K beads are dissolved in the lysis buffer in the first bead well 121, and the internal control beads are dissolved in the lysis buffer in the second bead well 122. At this time, mixing may be performed to dissolve the beads in the first bead well 121 and second bead well 122. For example, the apparatus for detecting a target analyte may apply heat to increase the temperature of the first bead well 121 and second bead well 122 to a predetermined temperature, and perform agitation with the pipette or give vibration in the detection apparatus to cause convection.

Then, the robot may move the pipette to transfer the lysis buffer from the lysis buffer well 114 to the processing well 141, transfer the solution in which the proteinase K beads are dissolved from the first bead well 121 to the processing well 141 and transfer the solution in which the internal control beads are dissolved from the second bead well 122 to the processing well 141.

Next, the robot may move the pipette to transfer the crude sample from the first sample well 101 to the second sample well 102. At this time, the pipette tip may be lowered to penetrate a sealing membrane for sealing the first sample well 101 and a sealing membrane for sealing the second sample well 102.

Then, the robot may move the pipette to draw up the filtered sample from the sample drawing well 103. At this time, the pipette tip may be lowered to penetrate a sealing membrane for sealing the sample drawing well 103.

For example, a negative pressure supplied from a pump of the apparatus for detecting a target analyte is delivered through a pipette, and the pipette may suck the sample from the sample drawing well 103 or second sample well 102 by a negative pressure. The sample in the second sample well 102 may pass through a filter by a negative pressure created by the pipette which enters the sample drawing well 103 and flow into the pipette tip.

The pumping process as described above may be repeated several times to suck the filtered sample into the pipette tip.

Next, the robot may move the pipette to transfer the filtered sample from the sample drawing well 103 to the processing well 141. Mixing may be performed in the processing well 141 for a predetermined period of time. For example, the apparatus for detecting a target analyte may perform agitation with the pipette or give vibration in the detection apparatus to cause convection.

Then, the apparatus for detecting a target analyte may apply heat to increase the temperature of the processing well 141 to a predetermined temperature. For example, the apparatus may apply heat to increase the temperature to 65° C., which is the extraction temperature, maintain the temperature at 65° C. for 120 seconds and stop heat application.

Next, the robot may move the pipette to transfer the mixed sample solution in an amount to be determined from the processing well 141 to the third bead well 123 in which magnetic beads are contained. At this time, the pipette tip may be lowered to penetrate a sealing membrane for sealing the third bead well 123.

Then, mixing may be performed to dissolve the beads in the third bead well 123. For example, the apparatus for detecting a target analyte may apply heat to increase the temperature of the third bead well 123 to a predetermined temperature, and perform agitation with the pipette or give vibration in the detection apparatus to cause convection.

Next, the robot may move the pipette to transfer the magnetic bead solution in an amount to be determined from the third bead well 123 to the processing well 141.

Next, the robot may move the pipette to transfer the binding buffer from the binding buffer well 114 in which the binding buffer is contained to the processing well 141. At this time, the pipette tip may be lowered to penetrate a sealing membrane for sealing the binding buffer well 114.

Then, mixing may be performed in the processing well 141 for a predetermined period of time. For example, the apparatus for detecting a target analyte may perform agitation with the pipette or give vibration in the detection apparatus to cause convection.

Then, the apparatus for detecting a target analyte may apply a magnetic field to the processing well 141 for a predetermined period of time for magnetization. At this time, the magnetic beads may stick to the inner wall of the processing well 141.

Next, the robot may move the pipette to transfer the supernatant from the processing well 141 to the waste well 104.

Then, the apparatus for detecting a target analyte may remove the magnetic field applied to the processing well 141 for demagnetization. At this time, the magnetic beads may come off from the inner wall of the processing well 141.

Next, the robot may move the pipette to transfer the cleaning buffer from the first buffer well 115 in which the cleaning buffer is contained to the processing well 141. At this time, the pipette tip may be lowered to penetrate a sealing membrane for sealing the first buffer well 115. Then, mixing may be performed in the processing well 141 for a predetermined period of time. For example, the apparatus for detecting a target analyte may perform agitation with the pipette or give vibration in the detection apparatus to cause convection.

Then, the apparatus for detecting a target analyte may apply a magnetic field to the processing well 141 for a predetermined period of time for magnetization. At this time, the magnetic beads may stick to the inner wall of the processing well 141.

Next, the robot may move the pipette to transfer the supernatant from the processing well 141 to the waste well 104.

Then, the robot may move the pipette to eject the first pipette tip to the first pipette tip holding hole 131.

Then, the apparatus for detecting a target analyte may remove the magnetic field applied to the processing well 141 for demagnetization. At this time, the magnetic beads may come off from the inner wall of the processing well 141.

Next, the robot may move the pipette to be coupled to a second pipette tip held in a second pipette tip holding hole 132.

Then, the robot may move the pipette to transfer the cleaning buffer from the second buffer well 116 in which the cleaning buffer is contained to the processing well 141. At this time, the pipette tip may be lowered to penetrate a sealing membrane for sealing the second buffer well 116.

Then, mixing may be performed in the processing well 141 for a predetermined period of time. For example, the apparatus for detecting a target analyte may perform agitation with the pipette or give vibration in the detection apparatus to cause convection.

Then, the apparatus for detecting a target analyte may apply a magnetic field to the processing well 141 for a predetermined period of time for magnetization. At this time, the magnetic beads may stick to the inner wall of the processing well 141.

Next, the robot may move the pipette to transfer the supernatant from the processing well 141 to the waste well 104.

Then, the robot may move the pipette to eject the second pipette tip to the second pipette tip holding hole 132.

Then, the apparatus for detecting a target analyte may remove the magnetic field applied to the processing well 141 for demagnetization. At this time, the magnetic beads may come off from the inner wall of the processing well 141.

Then, the apparatus for detecting a target analyte may heat the processing well 141 and circulate the magnetic beads to perform dry extraction. The circulation of magnetic beads may be performed by movement of polarity of the magnets or movement of the magnets around the processing well 141.

Next, the robot may move the pipette to be coupled to a third pipette tip held in a third pipette tip holding hole 133.

Then, the robot may move the pipette to transfer the elution buffer in an amount to be determined from the elution buffer wall 112 in which the elution buffer is contained to the processing well 141. At this time, the pipette tip may be lowered to penetrate a sealing membrane for sealing the elution buffer well 112.

Then, mixing may be performed in the processing well 141 for a predetermined period of time. For example, the apparatus for detecting a target analyte may perform agitation with the pipette or give vibration in the detection apparatus to cause convection.

Then, the apparatus for detecting a target analyte may apply heat to increase the temperature of the processing well 141 to a predetermined temperature. For example, the apparatus may apply heat to increase the temperature to 65° C., which is the extraction temperature, maintain the temperature at 65° C. for 300 seconds and stop heat application.

Then, the apparatus for detecting a target analyte may apply a magnetic field to the processing well 141 for a predetermined period of time for magnetization. At this time, the magnetic beads may stick to the inner wall of the processing well 141.

Next, the robot may move the pipette to eject the third pipette tip to the third pipette tip holding hole 133, and move the pipette to be coupled to a fourth pipette tip held in a fourth pipette tip holding hole 134.

Then, the robot may move the pipette to transfer the solution in an amount to be determined from the processing well 141 to each of the detection wells 151-156. At this time, the pipette tip may be lowered to penetrate sealing membranes for sealing the detection wells 151-156.

Each of the detection wells 151-156 may contain a lyophilized mastermix. The solution injected into each of the detection wells 151-156 by the fourth pipette tip may move to each detection site while the mastermix is dissolved in each of the detection wells 151-156. Here, the detection wells and the detection sites are connected, respectively, via a detection channel 201.

Next, the robot may move the pipette to eject the fourth pipette tip to the fourth pipette tip holding hole 134.

Finally, the apparatus for detecting a target analyte may perform a PCR process to detect the target analyte.

Hereinafter, a process for drawing a determined amount of a sample in the first sample containing well 101 using a pipette having a pipette tip mounted thereon and transferring the sample to the second sample containing well 102, and drawing the filtered sample in the sample drawing well 103 will be described in detail. The second sample containing well 102 is referred to as the sample containing part, and the sample drawing well 103 is referred to as the sample drawing part.

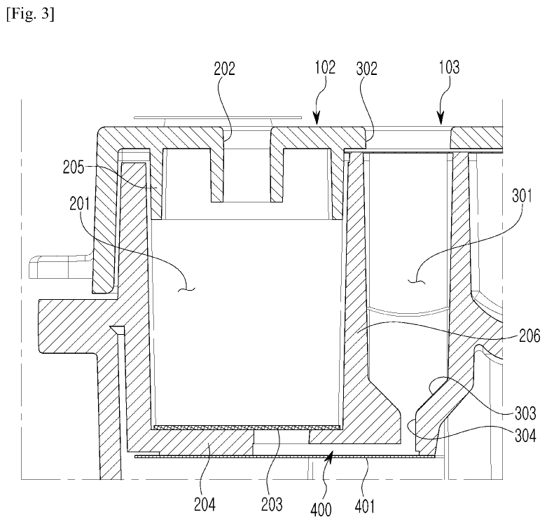

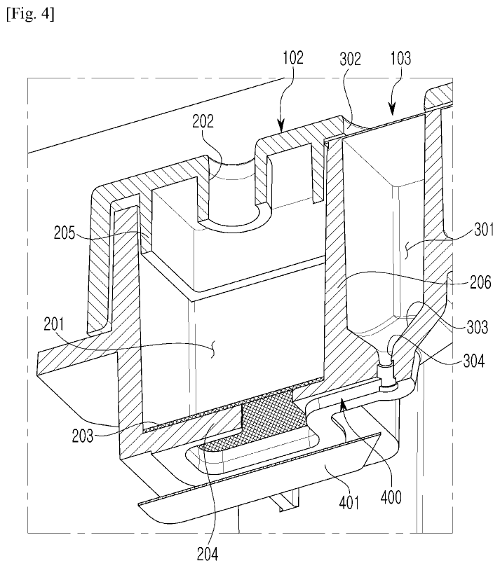

FIG. 3 is a cross-sectional view taken along line A-A in FIG. 2, and FIG. 4 is an exploded perspective view of FIG. 3.

Referring to FIG. 3 and FIG. 4, a cartridge 10 for detecting a target analyte comprises a sample containing part 102 defining a first chamber 201 and a sample drawing part 103 defining a second chamber 301.

The cartridge 10 may further comprise a sample flowing channel 400 via which the sample containing part 102 and the sample drawing part 103 are in fluid communication with each other. The cartridge may further comprise a filter member 203 for filtering a sample contained in the first chamber 201. The filter member 203 may be installed in the sample containing part 102 or the sample flowing channel 400.

The sample containing part 102 has a first pipette tip entrance 202 on an upper part thereof, and a lower part of the sample containing part is in communication with the sample flowing channel 400. The sample containing part 102 may further comprise a filter member 203. The filter member 203 may be spaced away from a bottom surface of the sample containing part 102. In the sample containing part 102, the sample before filtration may be above the filter member 203, and the sample after filtration may be below the filter member 203.

The sample flowing channel 400 may pass through a separation wall 206 between the first chamber 201 and the second chamber 301 or pass below the separation wall 206. The sample flowing channel 400 may have a small diameter within a range in which a flow resistance of a sample is below a threshold value. As the sample flowing channel 400 has a small diameter, the flow rate of the sample that passes through the sample flowing channel 400 may increase.

The inlet of the sample flowing channel 400 may have an opening cross-sectional area narrowing in a flow direction of a sample. For example, both sides of the inlet of the sample flowing channel 400 may be curved. The curved shape of the inlet of the sample flowing channel 400 may avoid a vortex that can occur around the inlet of the sample flowing channel 400 and reduce a flow resistance of the sample.

The sample drawing part 103 has a second pipette tip entrance 302 on an upper part thereof, and a lower part of the sample drawing part is in communication with the sample flowing channel 400. The sample drawing part 103 may have a pipette tip support part 303 for supporting the end of the pipette tip.

The pipette tip support part 303 may have a hole that is in communication with an outlet of the sample flowing channel 400. The hole formed in the pipette tip support part 303 may be a pipette tip support hole 304 which part of the end of the pipette tip is inserted into or in close contact with. And the pipette tip support hole 304 may be the outlet of the sample flowing channel 400.

And an inlet of the pipette tip and an inside of the sample drawing part 103 may be fluidly disconnected from each other in a state where the pipette tip is inserted into the pipette tip support hole 304 or is in close contact with the pipette tip support hole 304. And then, the negative pressure delivered through the pipette tip is delivered directly to the sample flowing channel 400.

Also, the pipette tip support part 303 may have a sloping surface sloping downwards towards the pipette tip support hole 304. For example, the sloping surface of the pipette tip support part 303 may have a conical shape around the pipette tip support hole 304.

The pipette tip entering through the second pipette tip entrance 302 may be in close contact with an outlet of the sample flowing channel 400. For example, the pipette tip may be supported on the pipette tip support part 303 in a state where part of the end of the pipette tip is inserted into the pipette tip support hole 304. A negative pressure delivered through the pipette tip may be delivered directly to the sample flowing channel 400 that is in communication with the pipette tip support hole 304. A filtered sample in the sample containing part 102 that is in communication with the sample flowing channel 400 may flow into the pipette tip by a negative pressure delivered through the sample flowing channel 400. Thus, the filtered sample in the sample containing part 102 may move into the pipette tip supported on the pipette tip support part 303.

As such, a negative pressure delivered through the pipette tip is used to suck the sample, thus obtaining the filtered sample more quickly.

It takes a certain amount of time or more for a sample before filtration in the sample containing part 102 to pass through the filter member 203 by spreading and gravity without an external force. As the filtration performance of the filter member 203 gets better, it takes more time for the sample to pass through the filter member 203.

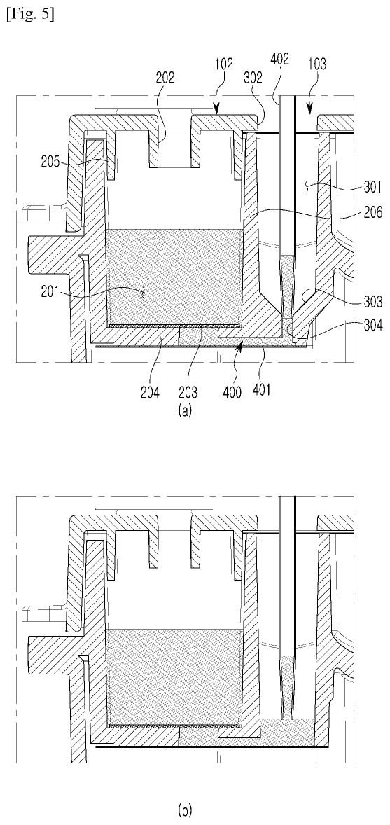

Hereinafter, a difference in suction time and amount of a sample that is sucked depending on whether there are a pipette tip support part 303 and a pipette tip support hole 304 will be described referring to FIG. 5.

FIG. 5 shows a difference in sample suction depending on whether there is a pipette tip support hole 304. Specifically, (a) of FIG. 5 shows a state where a pipette tip 402 is supported while being inserted into a pipette tip support hole 304 in a cartridge 10 according to an embodiment of the present disclosure, and (b) of FIG. 5 shows a state where a pipette tip 402 is close to the bottom surface of a sample drawing part 103 in a cartridge 10 according to a comparative example.

Referring to (a) of FIG. 5, in the cartridge 10 for detecting a target analyte according to an embodiment of the present disclosure, part of the end of the pipette tip 402 is inserted into the pipette tip support hole 304 arranged at the end of the outlet of the sample flowing channel 400, applying a negative pressure delivered through the pipette tip 402 directly to a first chamber 201 of the sample containing part 102. The negative pressure allows a sample before filtration which is above the filter member 203 to pass through the filter member 203 more quickly and flow into the pipette tip 402 via the sample flowing channel 400.

The sample may continuously flow within a volume range of the pipette tip, irrespective of the sample level in the sample containing part 102. For example, the sample filtered by passing through the filter member 203 may all flow into the pipette tip 402 while leaving no debris.

By comparison, referring to (b) of FIG. 5, in the case where the cartridge has no pipette tip support part and pipette tip support hole, as in the comparative example, in order to draw up a sample into the pipette tip 402 in the sample drawing part 103, a researcher should wait until the sample filtered by passing through the filter member 203 flows through the sample flowing channel 400 and fills the sample drawing part 103 to a certain level or higher. The sample only in a volume corresponding to the depth of the end of the pipette tip 402 that is sunk into the sample may flow into the pipette tip 402.

The sample that is below the end of the pipette tip 402 even after a long time is left over as debris.

Referring to FIG. 3 and FIG. 4 again, the sample containing part 102 may further comprise a spacer 204 for supporting the filter member 203. For example, the spacer 204 may be a support step extending from a side surface or bottom surface of the sample containing part 102, and supports part of the cross-sectional area of the filter member 203 to prevent the filter member 203 from being deformed due to a negative pressure and sticking to the bottom surface of the sample containing part 102.

At this time, if the spacer 204 has a greater area, the degree of deformation of the filter member 203 may be lowered, but the flow rate of the sample that passes through the filter member 203 may be reduced. If the spacer 204 has a smaller area, the reduction of the flow rate of the sample that passes through the filter member 203 may be prevented, but the degree of deformation of the filter member 203 may be raised. The area that supports the filter member 203, shape and location of the spacer 204 may be prepared to prevent the deformation of the filter member 203 and secure a high flow rate of the sample.

The spacer 204 may be spaced away from the inlet of the sample flowing channel 400 to prevent the occurrence of a vortex around the inlet of the sample flowing channel 400.

The sample containing part 102 may be formed integrally with the sample drawing part 103. For example, the sample containing part 102 and the sample drawing part 103 may be formed by processing a block. The sample flowing channel 400 via which the sample containing part 102 and the sample drawing part 103 are in communication may also be formed by processing the block.

The area in the bottom surface of the block where the sample flows may be opened and sealed with a sealing member 401. This structure may facilitate the formation of the sample flowing channel 400.

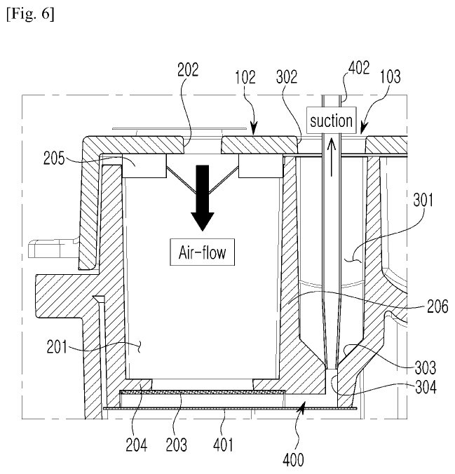

FIG. 6 is a cross-sectional view illustrating a cartridge according to another embodiment of the present disclosure.

A filter member 203 may be attached to a bottom surface of a spacer 204 or may be mechanically coupled thereto. The location change of the filter member 203 to below the spacer 204 may facilitate the installation and exchange of the filter member 203. That is, the filter member 203 may be mounted and exchanged through the opening in the bottom surface of the block, which saves labor hours.

The above-described description of the present disclosure is intended for illustration, and a person having ordinary knowledge in the art to which the present disclosure pertains will understand that the present disclosure may be easily modified and changed in other various forms without departing from the essential features of the present disclosure. Thus, the embodiments of the present disclosure are for illustrative purposes only, and the scope of the present disclosure is not limited to the present disclosure. It should be construed that the protection scope of the present disclosure is defined by the accompanying claims, and all equivalents fall within the scope of the present disclosure.

1. A cartridge for detecting a target analyte, comprising:
a sample containing part in which a sample is receivable;
a sample drawing part in which the sample is drawn up into a pipette tip; and
a sample flowing channel via which the sample containing part and the sample drawing part are in fluid communication with each other,
wherein a sample flows into a pipette tip from the sample containing part via the sample flowing channel by a negative pressure delivered through the pipette tip entering the sample drawing part in a state where the pipette tip is in close contact with an outlet of the sample flowing channel.

2. The cartridge of claim 1, wherein the sample flows directly into the pipette tip from the sample flowing channel.

3. The cartridge of claim 1, wherein the sample drawing part has a pipette tip support part for supporting the end of the pipette tip.

4. The cartridge of claim 3, wherein the pipette tip support part has a pipette tip support hole where the end of the pipette tip is inserted into or in close contact with.

5. The cartridge of claim 4, wherein the pipette tip support part has a sloping surface sloping downwards towards the pipette tip support hole.

6. The cartridge of claim 5, wherein the sloping surface of the pipette tip support part has a conical shape.

7. The cartridge of claim 4, wherein the pipette tip support hole is the outlet of the sample flowing channel.

8. The cartridge of claim 4, wherein an inlet of the pipette tip and an inside of the sample drawing part are fluidly disconnected from each other in a state where the pipette tip is inserted into the pipette tip support hole or is in close contact with the pipette tip support hole.

9. The cartridge of claim 4, wherein the negative pressure delivered through the pipette tip is delivered directly to the sample flowing channel in a state where the pipette tip is inserted into the pipette tip support hole or is in close contact with the pipette tip support hole.

10. The cartridge of claim 1, wherein the sample flowing channel fluidly connects a lower part of the sample containing part with a lower part of the sample drawing part.

11. The cartridge of claim 10, wherein the sample drawing part has a pipette tip support hole where the end of the pipette tip is inserted into or in close contact with, and the sample flowing channel is fluidly coupled to the pipette tip support hole.

12. The cartridge of claim 1, wherein the sample containing part or the sample flowing channel further comprises a filter member.

13. The cartridge of claim 12, wherein the filter member comprises a part spaced away from a bottom surface of the sample containing part by a certain distance.

14. The cartridge of claim 12, wherein the sample containing part has a pipette tip entrance that is prepared as a one-way valve.

15. The cartridge of claim 14, wherein when a pipette leaves the pipette tip entrance formed in the sample containing part, the one-way valve is closed to fluidly disconnect an inside of the sample containing part from an outside thereof.

16. The cartridge of claim 1, further comprising a separation wall for dividing a first chamber of the sample containing part and a second chamber of the sample drawing part, wherein the sample flowing channel passes through the separation wall or passes below the separation wall.

17. The cartridge of claim 16, further comprising a filter member disposed in front of an inlet of the sample flowing channel of the first chamber.

18. The cartridge of claim 17, wherein the filter member comprises a part spaced away from a bottom surface of the first chamber by a certain distance, and the sample flowing channel is in fluid communication with a space below the filter member.

19. The cartridge of claim 18, wherein the first chamber further comprises a spacer for supporting the filter member.

20. The cartridge of claim 19, wherein in the filter member, an area that is supported on the spacer is smaller than an area that is not supported on the spacer.

21. The cartridge of claim 19, wherein the spacer extends from a surface of the first chamber except from a separation wall in which an inlet of the sample flowing channel is formed.

22. The cartridge of claim 16, wherein a width of the sample flowing channel is smaller than a width of the separation wall, and an inlet of the sample flowing channel becomes narrower in a flow direction of a fluid.

23. The cartridge of claim 17, further comprising a housing comprising the first chamber, the second chamber and the separation wall, wherein a lower surface of the first chamber and the sample flowing channel is opened, and a housing sealing member for sealing the opened lower surface of the housing.

24. The cartridge of claim 23, wherein the housing sealing member is an elastically deformable membrane.

25. A cartridge for detecting a target analyte, comprising a body part including a pretreatment area having a plurality of wells, for pretreating a sample, and a detection area for detecting whether a target analyte is present in the sample pretreated in the pretreatment area,
wherein the body part comprises:
a sample containing well in which a crude sample is receivable, the sample containing well having a filter member;
a sample drawing well in which the sample is drawn up into a pipette tip;
a plurality of buffer wells in which buffers are receivable;
at least one reagent well in which reagent used for extraction or detection is receivable;
a processing well in which mixing of different fluids or extraction occurs; and
at least one detection well which is in communication with the pretreatment area via a channel,
wherein the sample containing well and the sample drawing well are in fluid communication with each other via a sample flowing channel and
wherein the sample flows directly into a pipette tip from the sample containing well via the sample flowing channel by a negative pressure delivered through the pipette tip entering the sample drawing well.

26. The cartridge of claim 25, further comprising a sample inlet well in which the crude sample comes in, and a waste well in which debris is stored.

27. The cartridge of claim 25, wherein the pipette tip entering the sample drawing well sucks a sample filtered by passing through the filter member from the sample containing well in which the sample is receivable.
